(19) United States
(12) Patent Application Publication
(10) Pub. No.: US 2017/0015975 A1
(43) Pub. Date: Jan. 19, 2017

(54) CELL MODULATION NANOCOMPOSITION, AND METHODS OF USE

(71) Applicant: NVIGEN, INC., Sunnyvale, CA (US)

(72) Inventors: Aihua FU, Sunnyvale, CA (US); Hua ZHOU, Sunnyvale, CA (US)

(21) Appl. No.: 15/117,729

(22) PCT Filed: Feb. 10, 2015
(86) PCT No.: PCT/US2015/015080

§ 371 Date: Aug. 10, 2016

Related U.S. Application Data
(60) Provisional application No. 61/938,103, filed on Feb. 10, 2014.

(51) Int. Cl. C12N 5/0783; C07K 16/30; C12N 5/00; C07K 16/28

(52) U.S. Cl. CPC C12N 5/0636; C07K 16/2809; C07K 16/2818; C07K 16/30; C07K 16/2803; C12N 5/0006; C12N 2535/00

(57) ABSTRACT

A nanocomposition for modulating cell behaviors and methods of uses thereof. The nanocomposition comprises a nanostructure comprising at least one nanoparticle and at least one cell-modulating agent operably linked to the nanostructure. The cell-modulating agent can interact with a molecule on the surface of a cell, wherein the interaction between the cell-modulating agent and the molecule modulates a behavior of the cell, or purify and concentrate a cell population.

### CROSS-REFERENCE TO RELATED APPLICATIONS

The present application is the National Stage of International Application PCT/US2015/015080, filed Feb. 10, 2015, which claims the benefit of U.S. Provisional Application Ser. No. 61/938,103, filed Feb. 10, 2014, which are incorporated herein by reference in their entirety.

### FIELD OF THE INVENTION

The present invention generally relates to using nanocomposition to modulate cell behavior.

### BACKGROUND OF THE INVENTION

Modern cell therapies often involve modulating cell behaviors in vivo or in vitro, such as stimulating cell proliferation, inducing cell differentiation and guiding cell migration. The current strategies and approaches to modulating cell behaviors, however, are limited. The application of nanotechnology in biomedical research represents a fascinating new outlook to create interesting and innovative tools based on modification at nanoscale level. Therefore, there are needs to develop and use nanotechnology of nanoparticles in modulating cell behavior.

### BRIEF SUMMARY OF THE INVENTION

The present disclosure provides nanocompositions and methods or uses of such nanocompositions. The nanocompositions can be used to modulate cell behaviors, such as cell proliferation, cell differentiation, cell activation, and obtaining a pure cell population in a concentration controllable manner.

One aspect of the present disclosure provides a nanocomposition for cell enrichment and modulation. The nanocomposition comprises a nanostructure and at least one cell-modulating agent operably linked to the nanostructure. The cell-modulating agent is capable of interacting with a molecule on the surface of a cell.

In some embodiments, the nanostructures in the nanocomposition comprises a magnetic material. In some embodiments, the magnetic material is a ferromagnetic, ferromagnetic, paramagnetic, or superparamagnetic material. In some embodiments, the magnetic material is superparamagnetic iron oxide (SPIO).

In some embodiments, the nanostructures in the nanocomposition have a silanization coating on a surface of the nanostructures.

In some embodiments, the nanostructures in the nanocomposition have a diameter ranging from 1 nm to 500 nm.

In some embodiments, the cell-modulating agent operably linked to the nanostructure comprises an antibody specifically recognizes the molecule on the surface of the cell. In some embodiments, the cell-modulating agent is selected from the group consisting of an anti-CD3 antibody, an anti-CD28 antibody an anti-CD81 antibody and any combination thereof.

In some embodiments, the cell-modulating agent is a ligand of a receptor on the surface of the cell. In some embodiments, the cell-modulating agent comprises a stimulatory form of a natural ligand for CD28 selected from the group consisting of B7-1 and B7-2.

In some embodiments, the cell-modulating agent is selected from a group consisting of a CD137 antibody, a CD137 ligand protein, a IL-15 protein, and a IL-15 receptor antibody.

In some embodiments, the cell-modulating agent is a vaccine.

In certain embodiments, the cell-modulating agent interacts with the cell so as to enrich a population of said cells or modulate a behavior of the cell. In some preferred embodiments, the behavior of the cell is transformation, proliferation, re-programming, differentiation or migration.

In certain embodiments, the cell can be used for therapy. In some preferred embodiments, the cell is capable of producing a chimeric antigen receptor.

In some embodiments, the cell whose behavior is modulated is a T cell. In some embodiments, the cell is a NK cell.

In some embodiments, the cell whose behavior is modulated is a stem cell. In some embodiments, the cell is an embryonic stem cell.

In some embodiments, the usability of the cells is because of their purity.

In some embodiments, the nanocomposition further comprises a detectable label. In some embodiments, the detectable label is a fluorescent molecule, a chemo-luminescent molecule, a bio-luminescent molecule, a radioisotope, a MM contrast agent, a CT contrast agent, an enzyme-substrate label, or a coloring agent.

In another aspect, the present disclosure provides a method for modulating the behavior of a cell by contacting the cell with at least one cell-modulating agent operably linked to a nanostructure. The cell-modulating agent interacts with a molecule on the surface of the cell, and the interaction between the cell-modulating agent and the molecule modulates the behavior of the cell. In some preferred embodiments, the method further comprises enriching a population of said cell.

Another aspect of the present invention relates to a method for treating a disease in a subject. The method comprises contacting a cell with at least one cell-modulating agent operably linked to a nanostructure. The cell-modulating agent interacts with a molecule on the surface of the cell. The interaction between the cell-modulating agent and the molecule modulates a behavior of the cell. And modulated cells are then administered to the subject.

### BRIEF DESCRIPTION OF THE FIGURES

FIG. 1 . . . Stimulation of CD4+ T cells using anti-CD3/anti-CD28 antibody conjugated nanocomposition.

FIG. 2. Isolation and identification of circulating tumor cells using nanocomposition.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, solid state chemistry, inorganic chemistry, organic chemistry, physical chemistry, analytical chemistry, materials chemistry, biochemistry, biology, molecular biology, recombinant DNA techniques, pharmacology, imaging, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

Nanocomposition

One aspect of the present disclosure provides a nanocomposition for cell enrichment and modulation comprising a nanostructure and at least one cell-modulating agent operably linked to the nanostructure, wherein the cell-modulating agent is capable of interacting with a molecule on the surface of a cell.

Nanostructure

The term “nanostructure” as used herein, refers to a particle having a diameter ranging from about 1 nm to about 1500 nm (e.g. from 1 nm to 1200 nm, from 1 nm to 1000 nm, from 1 nm to 800 nm, from 1 nm to 500 nm, from 1 nm to 400 nm, etc.). In certain embodiments, the nanostructure comprises a single particle or a cluster of particles. In certain embodiments, the nanostructure comprises a core nanoparticle and a coating. The core nanoparticle can be a single or a cluster of particles. The coating can be any coating known in the art, for example, a polymer coating such as polyethylene glycol, silane, and polysaccharides (e.g. dextran and its derivatives).

In some embodiments, the nanostructures provided herein contain a magnetic material. Suitable magnetic materials include, for example, ferrimagnetic or ferromagnetic materials (e.g., iron, nickel, cobalt, some alloys of rare earth metals, and some naturally occurring minerals such as lodestone), paramagnetic materials (such as platinum, aluminum), and superparamagnetic materials (e.g., superparamagnetic iron oxide or SPIO).

The magnetic material has magnetic property which allows the nanostructure to be pulled or attracted to a magnet or in a magnetic field. Magnetic property can facilitate manipulation (e.g., separation, purification, or enrichment) of the nanostructures using magnetic interaction. The magnetic nanostructures can be attracted to or magnetically guided to an intended site when subject to an applied magnetic field, for example a magnetic field from high-filed and/or high-gradient magnets. For example, a magnet (e.g., magnetic grid) can be placed in the proximity of the nanostructures so as to attract the magnetic nanostructures.

Any nanostructures having a magnetic property known in the art can be used. In certain embodiments, the nanostructure provided herein comprises a magnetic nanoparticle which comprises a magnetic material. For example, the magnetic nanoparticle of the nanostructure is a superparamagnetic iron oxide (SPIO) nanoparticle. The SPIO nanoparticle is an iron oxide nanoparticle, either maghemite (γ-Fe₂O₃) or magnetite (Fe₃O₄), or nanoparticles composed of both phases. The SPIO can be synthesized with a suitable method and dispersed as a colloidal solution in organic solvents or water. Methods to synthesize the SPIO nanoparticles are known in the art (see, for example, Morteza Mahmoudi et al, Superparamagnetic Iron Oxide Nanoparticles: Synthesis, Surface Engineering, Cytotoxicity and Biomedical Applications, published by Nova Science Pub Inc, 2011). In one embodiment, the SPIO nanoparticles can be made through wet chemical synthesis methods which involve co-precipitation of Fe and Fe salts in the presence of an alkaline medium. During the synthesis, nitrogen may be introduced to control oxidation, surfactants and suitable polymers may be added to inhibit agglomeration or control particle size, and/or emulsions (such as water-in-oil microemulsions) may be used to modulate the physical properties of the SPIO nanoparticle (see, for example, Jonathan W. Gunn, The preparation and characterization of superparamagnetic nanoparticles for biomedical imaging and therapeutic application, published by ProQuest, 2008). In another embodiment, the SPIO nanoparticles can be generated by thermal decomposition of iron pentacarbonyl, alone or in combination with transition metal carbonyls, optionally in the presence of one or more surfactants (e.g., lauric acid and oleic acid) and/or oxidatants (e.g., trimethylamine-N-oxide), and in a suitable solvent (e.g., dioctyl ether or hexadecane) (see, for example, US patent application PG Pub 20060093555). In another embodiment, the SPIO nanoparticles can also be made through gas deposition methods, which involves laser vaporization of iron in a helium atmosphere containing different concentrations of oxygen (see, Miller J. S. et al., Magnetism: Nanosized magnetic materials, published by Wiley-VCH, 2002). In certain embodiments, the SPIO nanoparticles are those disclosed in US patent application PG Pub 20100008862.

In certain embodiments, the nanostructure can further comprise a non-SIPO nanoparticle. The non-SPIO nanoparticles include, for example, metallic nanoparticles (e.g., gold or silver nanoparticles (see, e.g., Hiroki Hiramatsu, F. E. O., Chemistry of Materials 16, 2509-2511 (2004)), semiconductor nanoparticles (e.g., quantum dots with individual or multiple components such as CdSe/ZnS (see, e.g., M. Bruchez, et al, science 281, 2013-2016 (1998))), doped heavy metal free quantum dots (see, e.g., Narayan Pradhan et al, J. Am. chem. Soc. 129, 3339-3347 (2007)) or other semiconductor quantum dots); polymeric nanoparticles (e.g., particles made of one or a combination of PLGA (poly(lactic-co-glycolic acid) (see, e.g., Minsoung Rhee et al, Adv. Mater. 23, H79-H83 (2011)), PCL (polycaprolactone) (see, e.g., Marianne Labet et al, Chem. Soc. Rev. 38, 3484-3504 (2009)), PEG (poly ethylene glycol) or other polymers); siliceous nanoparticles; and non-SPIO magnetic nanoparticles (e.g., MnFe204 (see, e.g., Jae-Hyun Lee et al, Nature Medicine 13, 95-99 (2006)), synthetic antiferromagnetic nanoparticles (SAF) (see, e.g., A. Fu et al, Angew. Chem. Int. Ed. 48, 1620-1624 (2009)), and other types of magnetic nanoparticles). In certain embodiments, the non-SPIO nanoparticle is a colored nanoparticle, for example, a semiconductor nanoparticle such as a quantum dot.

The non-SPIO nanoparticles can be prepared or synthesized using suitable methods known in the art, such as for example, sol-gel synthesis method, water-in-oil micro-emulsion method, gas deposition method and so on. For example, gold nanoparticles can be made by reduction of chloroaurate solutions (e.g., HAuCl₄) by a reducing agent such as citrate, or acetone dicarboxulate. For another example, CdS semiconductor nanoparticle can be prepared from Cd(ClO₄)₂ and Na₂S on the surface of silica particles. For another example, II-VI semiconductor nanoparticles can be synthesized based on pyrolysis of organometallic reagents such as dimethyl cadmium and trioctylphosphine selenide, after injection into a hot coordinating solvent (see, e.g., Gunter Schmid, Nanoparticles: From Theory to Application, published by John Wiley & Sons, 2011). Doped heavy metal free quantum dots, for example Mn-doped ZnSe quantum dots can be prepared using nucleation-doping strategy, in which small-sized MnSe nanoclusters are formed as the core and ZnSe layers are overcoated on the core under high temperatures. For another example, polymeric nanoparticles can be prepared by emulsifying a polymer in a two-phase solvent system, inducing nanosized polymer droplets by sonication or homogenization, and evaporating the organic solvent to obtain the nanoparticles. For another example, siliceous nanoparticles can be prepared by sol-gel synthesis, in which silicon alkoxide precursors (e.g., TMOS or TEOS) are hydrolyzed in a mixture of water and ethanol in the presence of an acid or a base catalyst, the hydrolyzed monomers are condensed with vigorous stirring and the resulting silica nanoparticles can be collected. For another example, SAFs, a non-SPIO magnetic nanoparticle, can be prepared by depositing a ferromagenetic layer on each of the two sides of a nonmagnetic space layer (e.g., ruthenium metal), along with a chemical etchable copper release layer and protective tantalum surface layers, using ion-bean deposition in a high vacuum, and the SAF nanoparticle can be released after removing the protective layer and selective etching of copper.

The size of the nanoparticles ranges from 1 nm to 100 nm in size (preferable 1-50 nm, 2-40 nm, 5-20 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm in size). The size of nanoparticles can be controlled by selecting appropriate synthesis methods and/or systems. For example, to control the size of nanoparticles, synthesis of nanoparticles can be carried out in a polar solvent which provides ionic species that can adsorb on the surface of the nanoparticles, thereby providing electrostatic effect and particle-particle repulsive force to help stabilize the nanoparticles and inhibit the growth of the nanoparticles. For another example, nanoparticles can be synthesized in a micro-heterogeneous system that allows compartmentalization of nanoparticles in constrained cavities or domains. Such a micro-heterogeneous system may include, liquid crystals, mono and multilayers, direct micelles, reversed micelles, microemulsions and vesicles. To obtain nanoparticles within a desired size range, the synthesis conditions may be properly controlled or varied to provide for, e.g., a desired solution concentration or a desired cavity range (a detailed review can be found at, e.g., Vincenzo Liveri, Controlled synthesis of nanoparticles in microheterogeneous systems, Published by Springer, 2006).

The shape of the nanoparticles can be spherical, cubic, rod shaped (see, e.g., A. Fu et al, Nano Letters, 7, 179-182 (2007)), tetrapod-shaped (see, e.g., L. Manna et al, Nature Materials, 2, 382-385 (2003)), pyramidal, multi-armed, nanotube, nanowire, nanofiber, nanoplate, or any other suitable shapes. Methods are known in the art to control the shape of the nanoparticles during the preparation (see, e.g., Waseda Y. et al., Morphology control of materials and nanoparticles: advanced materials processing and characterization, published by Springer, 2004). For example, when the nanoparticles are prepared by the bottom-up process (i.e. from molecule to nanoparticle), a shape controller which adsorbs strongly to a specific crystal plane may be added to control the growth rate of the particle.

A single nanostructure may comprise a single nanoparticle or a plurality or a cluster of mini-nanoparticles (A. Fu et al, J. Am. chem. Soc. 126, 10832-10833 (2004), J. Ge et al, Angew. Chem. Int. Ed. 46, 4342-4345 (2007), Zhenda Lu et al, Nano Letters 11, 3404-3412 (2011).). The mini-nanoparticles can be homogeneous (e.g., made of the same composition/materials or having same size) or heterogeneous (e.g., made of different compositions/materials or having different sizes). A cluster of homogeneous mini-nanoparticles refers to a pool of particles having substantially the same features or characteristics or consisting of substantially the same materials. A cluster of heterogeneous mini-nanoparticles refers to a pool of particles having different features or characteristics or consisting of substantially different materials. For example, a heterogeneous mini-nanoparticle may comprise a quantum dot in the center and a discrete number of gold (Au) nanocrystals attached to the quantum dot. When the nanoparticles are associated with a coating (as described below), different nanoparticles in a heterogeneous nanoparticle pool do not need to associate with each other at first, but rather, they could be individually and separately associated with the coating.

In certain embodiments, a nanostructure disclosed comprises a plurality of nanoparticles. For example, the nanostructure contains 2, 3, 4, 5, 6, 7, 8, 9, 10, 11, 12, 13, 14, 15, 16, 17, 18, 19, 20, 25, 30, 35, 40, 50, 60, 70, 80, 90, 100, 100s or 1000s nanoparticles.

In certain embodiments, the nanostructure provided herein further comprises a coating. At least one core nanoparticle can be embedded in or coated with the coating. Any suitable coatings known in the art can be used, for example, a polymer coating and a non-polymer coating. The coating interacts with the core nanoparticles through 1) intra-molecular interaction such as covalent bonds (e.g., Sigma bond, Pi bond, Delta bond, Double bond, Triple bond, Quadruple bond, Quintuple bond, Sextuple bond, 3c-2e, 3c-4e, 4c-2e, Agostic bond, Bent bond, Dipolar bond, Pi backbond, Conjugation, Hyperconjugation, Aromaticity, Hapticity, and Antibonding), metallic bonds (e.g., chelating interactions with the metal atom in the core nanoparticle), or ionic bonding (cation π-bond and salt bond), and 2) inter-molecular interaction such as hydrogen bond (e.g., Dihydrogen bond, Dihydrogen complex, Low-barrier hydrogen bond, Symmetric hydrogen bond) and non covalent bonds (e.g., hydrophobic, hydrophilic, charge-charge, or π-stacking interactions, van der Waals force, London dispersion force, Mechanical bond, Halogen bond, Aurophilicity, Intercalation, Stacking, Entropic force, and chemical polarity).

In certain embodiments, the coating comprises a low density, porous 3-D structure, as disclosed in U.S. Prov. Appl. 61/589,777 and U.S. patent application Ser. No. 12/460,007 (all references cited in the present disclosure are incorporated herein in their entirety).

The low density, porous 3-D structure refers to a structure with density much lower (e.g., 10s times, 20s times, 30s times, 50s times, 70s times, 100s times) than existing mesoporous nanoparticles (e.g., mesoporous nanoparticles having a pore size ranging from 2 nm to 50 nm). (A. Vincent, et. al., J. Phys. Chem. C, 2007, 111, 8291-8298; J. E. Lee, et. al, J. Am. Chem. Soc, 2010, 132, 552-557; Y.-S. Lin, et. al, J. Am. Chem. Soc, 2011, 133, 20444-20457; Z. Lu, Angew. Chem. Int. Ed., 2010, 49, 1862-1866.)

In certain embodiments, the low density, porous 3-D structure refers to a structure having a density of <1.0 g/cc (e.g., <100 mg/cc, <10 mg/cc, <5 mg/cc, <1 mg/cc, <0.5 mg/cc, <0.4 mg/cc, <0.3 mg/cc, <0.2 mg/cc, or <0.1 mg/cc) (for example, from 0.01 mg/cc to 10 mg/cc, from 0.01 mg/cc to 8 mg/cc, from 0.01 mg/cc to 5 mg/cc, from 0.01 mg/cc to 3 mg/cc, from 0.01 mg/cc to 1 mg/cc, from 0.01 mg/cc to 1 mg/cc, from 0.01 mg/cc to 0.8 mg/cc, from 0.01 mg/cc to 0.5 mg/cc, from 0.01 mg/cc to 0.3 mg/cc, from 0.01 mg/cc to 1000 mg/cc, from 0.01 mg/cc to 915 mg/cc, from 0.01 mg/cc to 900 mg/cc, from 0.01 mg/cc to 800 mg/cc, from 0.01 mg/cc to 700 mg/cc, from 0.01 mg/cc to 600 mg/cc, from 0.01 mg/cc to 500 mg/cc, from 0.1 mg/cc to 800 mg/cc, from 0.1 mg/cc to 700 mg/cc, from 0.1 mg/cc to 1000 mg/cc, from 1 mg/cc to 1000 mg/cc, from 5 mg/cc to 1000 mg/cc, from 10 mg/cc to 1000 mg/cc, from 20 mg/cc to 1000 mg/cc, from 30 mg/cc to 1000 mg/cc, from 30 mg/cc to 1000 mg/cc, from 30 mg/cc to 900 mg/cc, from 30 mg/cc to 800 mg/cc, or from 30 mg/cc to 700 mg/cc).

The density of 3-D structure can be determined using various methods known in the art (see, e.g., Lowell, S. et al, Characterization of porous solids and powders: surface area, pore size and density, published by Springer, 2004). Exemplary methods include, Brunauer Emmett Teller (BET) method and helium pycnometry (see, e.g., Varadan V. K. et al., Nanoscience and Nanotechnology in Engineering, published by World Scientific, 2010). Briefly, in BET method, dry powders of the testing 3-D structure is placed in a testing chamber to which helium and nitrogen gas are fed, and the change in temperature is recorded and the results are analyzed and extrapolated to calculate the density of the testing sample. In helium pycnometry method, dry powders of the testing 3-D structure are filled with helium, and the helium pressure produced by a variation of volume is studied to provide for the density. The measured density based on the dry power samples does not reflect the real density of the 3-D structure because of the ultralow density of the 3-D structure, the framework easily collapses during the drying process, hence providing much smaller numbers in the porosity measurement than when the 3-D structure is fully extended, for example, like when the 3-D structure is fully extended in a buffer solution. In certain embodiments, the density of the 3-D structure can be determined using the dry mass of the 3-D structure divided by the total volume of such 3-D structure in an aqueous solution. For example, dry mass of the core particles with and without the 3-D structure can be determined respectively, and the difference between the two would be the total mass of the 3-D structure. Similarly, the volume of a core particle with and without the 3-D structure in an aqueous solution can be determined respectively, and the difference between the two would be the volume of the 3-D structure on the core particle in an aqueous solution.

In certain embodiments, the porous nanostructure can be dispersed as multiple large nanoparticles coated with the 3-D structure in an aqueous solution, in such case, the total volume of the 3-D structure can be calculated as the average volume of the 3-D structure for an individual large nanoparticle multiplied with the number of the large nanoparticles. For each individual large nanoparticle, the size (e.g., radius) of the particle with 3-D structure can be determined with Dynamic Light Scattering (DLS) techniques, and the size (e.g., radius) of the particle core without the 3-D structure can be determined under Transmission Electron Microscope (TEM), as the 3-D structure is substantially invisible under TEM. Accordingly, the volume of the 3-D structure on an individual large nanoparticle can be obtained by subtracting the volume of the particle without 3-D structure from the volume of the particle with the 3-D structure.

The number of large nanoparticles for a given core mass can be calculated using any suitable methods. For example, an individual large nanoparticle may be composed of a plurality of small nanoparticles which are visible under TEM. In such case, the average size and volume of a small nanoparticle can be determined based on measurements under TEM, and the average mass of a small nanoparticle can be determined by multiplying the known density of the core material with the volume of the small particle. By dividing the core mass with the average mass of a small nanoparticle, the total number of small nanoparticles can be estimated. For an individual large nanoparticle, the average number of small nanoparticles in it can be determined under TEM. Accordingly, the number of large nanoparticles for a given core mass can be estimated by dividing the total number of small nanoparticles with the average number of small nanoparticels in an individual large nanoparticle. Alternatively, the low density, porous 3-D structure refers to a structure having 40%-99.9% (preferably 50% to 99.9%) of empty space or pores in the structure, where 80% of the pores having size of 1 nm to 500 nm in pore radius.

The porosity of the 3-D structure can be characterized by the Gas/Vapor adsorption method. In this technique, usually nitrogen, at its boiling point, is adsorbed on the solid sample. The amount of gas adsorbed at a particular partial pressure could be used to calculate the specific surface area of the material through the Brunauer, Emmit and Teller (BET) nitrogen adsorption/desorption equation. The pore sizes are calculated by the Kelvin equation or the modified Kelvin equation, the BJH equation (see, e.g., D. Niu et al, J. Am. chem. Soc. 132, 15144-15147 (2010)). The porosity of the 3-D structure can also be characterized by mercury porosimetry (see, e.g., Varadan V. K. et al, supra). Briefly, gas is evacuated from the 3-D structure, and then the structure is immersed in mercury. As mercury is non-wetting at room temperature, an external pressure is applied to gradually force mercury into the sample. By monitoring the incremental volume of mercury intruded for each applied pressure, the pore size can be calculated based on the Washburn equation.

Alternatively, the low density, porous 3-D structure refers to a structure that has a material property, that is, the porous structure (except to the core nanoparticle or core nanoparticles) could not be obviously observed or substantially transparent under transmission electron microscope, for example, even when the feature size of the 3-D structure is in the 10s or 100s nanometer range. The term “obviously observed” or “substantially transparent” as used herein means that, the thickness of the 3-D structure can be readily estimated or determined based on the image of the 3-D structure under TEM. The nanostructure (e.g., nanoparticles coated with or embedded in/on a low density porous 3-D structure) can be observed or measured by ways known in the art. For example, the size (e.g., radius) of the nanostructure with the 3-D structure can be measured using DLS methods, and the size (e.g., radius) of the core particle without the 3-D structure can be measured under TEM. In certain embodiments, the thickness of the 3-D structure is measured as 10s, 100s nanometer range by DLS, but cannot be readily determined under TEM. For example, when the nanostructures provided herein are observed under Transmission Electron Microscope (TEM), the nanoparticles can be identified, however, the low density porous 3-D structure can not be obviously observed, or is almost transparent. This distinguishes the low density porous 3-D structures from those reported in the art that comprise nanoparticles coated with crosslinked and size tunable 3-D structure, including the mesoporous silica nanoparticles or coating (see, e.g., J. Kim, et. al, J. Am. Chem. Soc, 2006, 128, 688-689; J. Kim, et. al, Angew. Chem. Int. Ed., 2008, 47, 8438-8441). This feature also indicates that the low density porous 3-D structure has a much lower density and/or is highly porous in comparison to other coated nanoparticles known in the art. The porosity of the 3-D structure can be further evaluated by the capacity to load different molecules (see, e.g., Wang L. et al, Nano Research 1, 99-115 (2008)). As the 3-D structure provided herein has a low density, it is envisaged that more payload can be associated with the 3-D structure than with other coated nanoparticles. For example, when 3-D structure is loaded with organic fluorophores such as Rhodamin, over 105 Rhodamin molecules can be loaded to 3-D structure of one nanoparticle.

In certain embodiments, the low density, porous 3-D structure is made of silane-containing or silane-like molecules (e.g., silanes, organosilanes, alkoxysilanes, silicates and derivatives thereof).

In certain embodiments, the silane-containing molecule comprises an organosilane, which is also known as silane coupling agent. Organosilane has a general formula of RₓSiY₍₄₋ₓ₎, wherein R group is an alkyl, aryl or organo functional group. Y group is a methoxy, ethoxy or acetoxy group, x is 1, 2 or 3. The R group could render a specific function such as to associate the organosilane molecule with the surface of the core nanoparticle or other payloads through covalent or non-covalent interactions. The Y group is hydro lysable and capable of forming a siloxane bond to crosslink with another organosilane molecule. Exemplary R groups include, without limitation, disulphidealkyl, aminoalkyl, mercaptoalkyl, vinylalkyl, epoxyalkyl, and methacrylalkyl, carboxylalkyl groups. The alkyl group in an R group can be methylene, ethylene, propylene, and etc. Exemplary Y groups include, without limitation, alkoxyl such as OCH₃, OC₂H₅, and OC₂H₄OCH₃. For example, the organosilane can be amino-propyl-trimethoxysilane, mercapto-propyl-trimethoxysilane, carboxyl-propyl-trimethoxysilane, amino-propyl-triethoxysilane, mercapto-propyl-triethoxysilane, carboxyl-propyl-triethoxysilane, Bis-[3-(triethoxysilyl) propyl]-tetrasulfide, Bis-[3-(triethoxysilyl) propyl]-disulfide, aminopropyltriethoxysilane, N-2-(aminoethyl)-3-amino propyltrimethoxysilane, Vinyltrimethoxysilane, Vinyl-tris(2-methoxyethoxy) silane, 3-methacryloxypropyltrimethoxy silane, 2-(3,4-epoxycyclohexy)-ethyl trimethoxysilane, 3-glycidoxy-propyltriethoxysilane, 3-isocyanatopropyltriethoxysilane, and 3-cyanatopropyltriethoxy silane.

Cell-Modulating Agent

The nanostructure is operably linked to at least one cell-modulating agent.

The term “operably linked” as used herein, includes embedding, incorporating, integrating, binding, attaching, combining, cross-linking, mixing, and/or coating the cell-modulating agent to the nanostructure. The cell-modulating agent can be operably linked to the nanostructure through non-covalent association (e.g., hydrogen bonds, ionic bonds, van der Waals forces, and hydrophobic interaction) or covalent binding. For example, the cell-modulating agent mixed with and/or incorporated onto the surface of the nanostructure, or can also be loaded to the pores of the nanostructure.

“Modulating,” “modulation” or “modulate” as used herein, means an alternation and/or regulation of a cell. The alternation and/or regulation of a cell can be determined by comparing the properties of a cell binding to the cell-modulating agent with that of a control (i.e., cells not binding to the cell-modulating agent). The alternation and/or regulation of the cell can be measured based on various properties of the cell, including without limitation, the number of the cells in the cell population, the morphology of the cell, the lineage/type of the cell (e.g., a transition from one cell type to another), the state of the cell (e.g., rearrange or recombination of DNA or chromosome, expression change of RNA or protein, secretion or trafficking of proteins), the mobility or migration of the cell. The alternation and/or regulation of a cell can be determined using suitable methods known in the art, including, for example, observation using microscopy, cell counting, cell sorting, immuno-histochemistry, immuno-cell-chemistry, PCR, northern-blot, southern blot, western-blot (see, e.g., Julio E. Celis et al., Cell Biology, A Laboratory Handbook (3rd Ed.)).

“Interact” or “bind” as used herein, means a non-random association between two molecules. The non-random association can be characterized by binding affinity (Kd), which is calculated as the ratio of dissociation rate to association rate (k_{off}/kₒₙ) when the binding between the two molecules reaches equilibrium. The dissociation rate (k_{off}) measured at the binding equilibrium may also be used when measurement of kₒₙ is difficult to obtain, for example, due to aggregation of one molecule. The binding affinity (e.g., Kd or k_{off}) between the cell-modulating agent and the molecule on the surface of a cell can be appropriately determined using suitable methods known in the art, including, for example, Biacore (see, for example, Murphy, M. et al, Current protocols in protein science, Chapter 19, unit 19.14, 2006) and Kinexa techniques (see, for example, Darling, R. J., et al, Assay Drug Dev. TechnoL, 2(6): 647-657 (2004)).

In some embodiments, the cell-modulating agent operably linked to the nanostructure comprises an antibody specifically recognizes the molecule on the surface of the cell. For one example, the cell-modulating agent comprises an anti-CD3 antibody. For another example, the cell-modulating agent comprises an anti-CD28 antibody. For another example, the cell-modulating agent comprises a CD137 antibody. For yet anther example, the cell-modulating agent is a IL-15 receptor antibody.

As used herein, the term “antibody” is intended to include polyclonal and monoclonal antibodies, chimeric antibodies, haptens and antibody fragments, and molecules which are antibody equivalents in that they specifically bind to an epitope on the antigen. The term “antibody” includes polyclonal and monoclonal antibodies of any isotype (IgA, IgG, IgE, IgD, IgM), or an antigen-binding portion thereof, including, but not limited to, F(ab) and Fv fragments such as sc Fv, single chain antibodies, chimeric antibodies, humanized antibodies, and a Fab expression library.

In some embodiments, the cell-modulating agent is a ligand of a receptor on the surface of the cell. For one example, the cell-modulating agent comprises a stimulatory form of a natural ligand for CD28 selected from the group consisting of B7-1 and B7-2. For another example, the cell-modulating agent is a CD137 ligand protein. For another example, the cell-modulating agent is a CD81 ligand protein. For another example, the cell-modulating agent is a IL-15 protein. For another example, the cell-modulating agent is a cytokine, including chemokines (e.g., CCL14, CCL19, CCL20, CCL21, CCL25, CCL27, CXCL12 and CXCL13, IL-1, TNF-alpha, LPS, CXCL-8, CCL2, CCL3, CCL4, CCL5, CCL11, CXCL10), interferons (e.g., INF-alpha, INF-beta, INF-gamma), interleukins (e.g., IL-1, IL-2, IL-3, IL-4, IL-5, IL-6, IL-7, IL-8, IL-9, IL-10, IL-11, IL-12, IL-13, IL-15, IL-17), lymphokines (e.g., IL-2, IL-3, IL-4, IL-5, IL-6, granular-macrophage CSF, INF-gamma), tumor necrosis factor (e.g., TNF-alpha, Lympahotoxin-alpha, gp39 (CD40L), CD27L, CD30L, FASL, 4-1BBL, OX40L, TNF-related spoptosis induicing ligand (TRAIL)). For another example, the cell-modulating agent is a hormone, including prolacin, vasopressin, oxytocin, atrial-natriuretic peptide (ANP), atrial natriuretic factor (ANF), glucagon, insulin, somatostatin, cholecystokinin, gastrin, leptin, Luteinizing hormone, follicle-stimulating hormone or thryroid-stimulating hormone. For yet another example, the cell-modulating agent is a growth factor, including Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumor necrosis factor-alpha(TNF-α), Vascular endothelial growth factor (VEGF), Wnt Signaling Pathway, placental growth factor (PGF), Fetal Bovine Somatotrophin (FBS), IL-1, IL-2, IL-3, IL-4, IL-5, IL-6, IL-7.

In certain embodiments, the cell-modulating agent is selected from the group consisting of an anti-CD3 antibody, an anti-CD28 antibody, an anti-CD81 antibody, a stimulatory form of a CD28 ligand, an anti-CD5 antibody, an anti-CD4 antibody, an anti-CD8 antibody, an anti-CTLA-4 antibody, an anti-PD-1 antibody, and anti-PD-L1 antibody, an anti-CD278 antibody, an anti-CD27L antibody, an anti-CD137 antibody, a CD137 ligand protein, an anti-CD30L antibody, an IL-2, an IL-2 receptor antibody, a IL-15 protein, a IL-15 receptor antibody, an IL-12, an IL-12 receptor antibody, an IL-1, an IL-1 receptor antibody, an IFN-gamma, an IFN-gamma receptor antibody, an TNF-alpha, an TNF-alpha receptor antibody, an IL-4, and IL-4 receptor antibody, an IL-10, an IL-10 receptor antibody and any combination thereof.

In some embodiments, the cell-modulating agent is a vaccine. A vaccine is a molecule that improves immunity to a particular disease. In some embodiments, the cell-modulating agent resembles a diseases-causing microorganism and is made from weakened or killed forms of the microbe, its toxins or one of its surface proteins. For example, the cell-modulating agent is a vaccine against adenovirus, anthrax, BCG live, diphtheria, tetanus toxoids, acelluar pertussis, haemophilus b, hepatitis A, hepatitis B, human papillomavirus, influenza A (H1N1), influenza virus, influenza A (HSN1), Japanese encephalitis virus, measles, mumps virus, rubella virus, meningococcal, plague, pheumococcal, poliovirus, rabies, rotavirus, smallpox, typhoid, varicella virus, yellow fever, zoster. In some embodiments, the cell-modulating agent is a cancer vaccine. For example, the cell-modulating agent is tumor antigens, i.e., proteins separated from cancer cells. For another example, the cell-modulating agent can be BiovaxID (treat follicular lymphoma), Provenge (treat prostate cancer), Tarmagens, melanoma-associated antigen 3 (MAGE-A3), PROSTVAC, CDX110, CDX1307, CDX1401, CimaVax-EGF (treat lung cancer), CV9104, Neuvenge, Neu Vax, Ax-37, ADXS11-001, ADXS31-001, ADXS31-164, GI-4000, GRNVAC1, GI6207, GI6301, IMA901, Stimuvax, Cvac, SCIB1.

Molecules on the Surface of a Cell

The cell-modulating agent can interact with a molecule on the surface of a cell. The molecule is present on the surface of a cell constitutively or transiently. In some embodiments, the molecule appears on the surface of a cell after the cell has been modulated by a nanocomposition as described herein.

In some embodiments, the molecule on the surface of a cell is a cell surface receptor. In some embodiments, the cell surface receptor is a specialized integral membrane protein that takes part in communication between the cell and the environment. In some embodiments, the molecule on the surface of the cell is a cytokine receptor, for example, interleukin receptor, erythorprietin receptor, GM-CSF receptor, G-CSF receptor, growth hormone receptor, prolactin receptor, oncostatin M receptor, leukemia inhibitory factor receptor, interferon alpha/beta receptor, interferon-gamma receptor, IL-1 receptor, CSF1, C-kit receptor, IL-18 receptor, CD27, CD30, CD40, CD120, lymphotoxin beta receptor, IL-8 receptor, IL-17 receptor, CCR1, CXCR4, MCAF receptor, NAP-2 receptor, TGF beta receptor. In some embodiments, the molecule on the surface of the cell is a growth factor receptor, for example, calcitonin receptor, calcitonin receptor like receptor, VEGF receptor, EGF receptor, FGF receptor, BMP receptor, BDNF receptor, erythropoietin receptor, GDNF receptor, G-CSF receptor, GM-CSF receptor, GDF receptor, HGF receptor, HDGF receptor, IGF receptor, NGF receptor, PDGF receptor, TPO receptor, TGF-alpha receptor, TGF-beta receptor). In some embodiments, the molecule on the surface of a cell is a hormone receptor, for example, insulin receptor, thyroid-stimulating hormone receptor, follicle-stimulating hormone receptor, leutinizing hormone receptor.

In certain preferred embodiments, the cell-modulating agent operably linked to the nanostructure comprises an antibody specifically binds to the cell-surface receptor as disclosed above.

In some embodiments, the molecule on the surface of a cell is a cell adhesion molecule. In some embodiments, the cell adhesion molecule is a protein located on the cell surface involved in binding with other cells or with the extracellular matrix, helping the cell stick to each other or its surroundings. In some embodiments, the molecule on the surface of a cell is a immunoglobulin superfamily cell adhesion molecule, for example, synaptic cell adhesion molecule, neural cell adhesion molecule, intercellular cell adhesion molecule, vascular cell adhesion molecule, platelet-endothelial cell adhesion molecule, L1 protein, CRL1, neurofascin, NrCAM, myelin-associated glycoprotein, CD22, CD83, CTX, junctional adhesion molecule, BT-IgSF, coxsackie virus and adenorvirus receptor, VSIG, ESAM, nectins, nextin-like molecules, CD2, CD48. In some embodiments, the molecule on the surface of a cell is a lymphocyte homing receptor, for example, CD34 and GLYCAM-1. In some embodiments, the molecule on the surface of a cell is an integrin. In some embodiments, the molecule on the surface of a cell is a cadherin. In some embodiments, the molecule on the surface of a cell is a selectin, for example, F-selectin, L-selectin, and P-selectin.

Cell and its Behaviors being Modulated

The interaction between the cell-modulating agent and the molecule on the surface of a cell modulates a behavior of the cell, triggering changes in the function or property of the cell. The cell includes both prokaryotic cells and eukaryotic cells. In some embodiments, the cell is an animal cell. In some preferred embodiments, the cell is a mammalian cell, for example, a mouse cell, a rat cell, a rabbit cell, a monkey cell, a human cell. The cell can be isolated and cultured in vitro, or present in vivo.

The cell can be any type exists in an organism of interest, for example, cells derived from endoderm (e.g., exocrine secretory cells and hormone secreting cells), cells derived from ectoderm (e.g., epithelial cells, neural cell), and cells derived from mesoderm (e.g., metabolism and storage cells, barrier function cells (lung cells, gut cells, exocrine gland cells), kidney cells, extracellular matrix cells, contractile cells (muscle cells), blood and immune system cells, germ cells, nurse cells). In some embodiments, the cell is an immune system cell, for example, T-cell, B-cell, natural killer (NK) cell, macrophage. In some preferred embodiments, the cell is a T-cell. In some preferred embodiments, the cell is a NK-cell.

In some embodiments, the cell is a stem cell, for example, embryonic stem cell, induced pluripotent stem cell, hematopoietic stem cell, mammary stem cell, intestinal stem cell, mesenchymal stem cell, endothelial stem cell, neural stem cell, neural crest stem cell.

The behavior of the cell that is modulated can be any function or property of the cell, including without limitation, cell proliferation, cell growth, cell differentiation, cell activation, cell transformation, cell migration, cell motility, cell mobility, cell apoptosis and cell adhesion, cell purity, and cell capability of use for therapy

In some preferred embodiments, the behavior of the cell being modulated is cell proliferation. For example, T-cell proliferation can be activated by administering an anti-CD3 antibody conjugated with a polymer backbone or microbead (see, U.S. Pat. No. 6,129,916). Similarly, T-cell proliferation can be activated by contacting the T cells in vitro with an anti-CD3 antibody and an anti-CD28 antibody, both of which are immobilized on a solid phase surface (see U.S. Pat. No. 6,352,694). T-cell proliferation can also be activated by contacting with an anti-CD3 antibody and a stimulatory form of a natural ligand for CD28, such as B7-1 and B7-2, wherein both anti-CD3 antibody and natural ligand for CD28 are immobilized on a solid phase surface (see U.S. Pat. No. 6,352,694). NK cell proliferation can be activated by contacting the NK cell with a CD137 ligand protein, a CD137 antibody, a IL-15 protein or an IL-15 receptor antibody, wherein the CD137 ligand protein, CD137 antibody, IL-15 protein or IL-15 receptor antibody is immobilized on a solid phase support (see U.S. Pat. No. 8,399,645).

In some preferred embodiments, the behavior of the cell being modulated is cell differentiation. In some embodiments, modulation of differentiation can be achieved by contacting the stem cell with a molecule that can induce the differentiation of the stem cell, wherein the molecule is operably linked to the nanostructure. For example, CD34 positive cells can be induced to differentiate into NK cells by administering IL-12 linked to a microbead.

In some preferred embodiments, the cells whose behavior is modulated can be used for therapy. In some embodiments, the cells could be further modified to express a certain protein for therapy. In some preferred embodiments, the cells are capable of producing a chimeric antigen receptor (CAR). Examples of chimeric antigen receptor are illustrated in U.S. Pat. No. 8,399,645 (anti-CD19 single chain variable fragment domain, 4-1BB signaling domain and CD3zeta signaling domain chimeric receptor); U.S. Pat. No. 5,686,281 (T-cell receptor CD28 signaling domain chimeric receptor); Geiger, T. L. et al., Blood 98: 2364-2371 (2001); Hombach, A. et al., J Immunol 167: 6123-6131 (2001) (CD28/CD3 zeta signaling receptor); Maher, J. et al. Nat Biotechnol 20: 70-75 (2002) (TCRzeta/CD28 recetpor); Haynes, N. M. et al., J Immunol 169: 5780-5786 (2002) (anti-carcinoembryonic antigen single chain variable fragment/CD28 zeta chimeric receptor); Haynes, N. M. et al., Blood 100: 3155-3163 (2002) (anti erbB2 single chain variable fragment/CD28/TCR zeta chimeric receptor); Till B. G. et al., Blood 119(17):3940-50 (2012) (CD20 specific CAR with CD28 and 4-1BB costimulatory domains); Haso W. et al., Blood 121(7):1165-74 (2013) (CD22 specific CAR). These references are herein incorporated into the specification.

Colored Nanostructure

The nanostructure provided herein can be colored or non-colored. “Colored” as used herein, means that the nanostructure is capable of generating a color signal under a suitable condition. For example, the colored nanostructure may emit a fluorescent color signal upon excitation with a light of a certain wavelength. The nanostructures may alternatively be non-colored. A non-colored nanostructure does not emit a color signal when subject to a condition that would otherwise induce a color signal for a colored nanostructure.

In certain embodiments, a colored nanostructure is bar-coded or associated with a detectable agent to show color. The term “bar-coding” or “bar-coded” or “IDed” means that the nanostructure is associated with a known code or a known label that allows identification of the nanostructure. “Code” as used herein, refers to a molecule capable of generating a detectable signal that distinguishes one bar-coded or IDed nanostructure from another. For example, the colored nanostructure may comprise a colored nanoparticle (e.g. a quantum dot) which emits a detectable color signal at a known wave length.

In certain embodiments, the characteristics or the identity of a bar-coded nanostructure is based on multiplexed optical coding system as disclosed in Han et al, Nature Biotechnology, Vol. 19, pp: 631-635 (2001) or U.S. patent application Ser. No. 10/185,226. Briefly, multicolor semiconductor quantum-dots (QDs) are embedded in the nanostructure. For each QD, there is a given intensity (within the levels of, for example. 0-10) and a given color (wavelength). For each single color coding, the nanostructure has different intensity of QDs depending on the number of QDs embedded therein. If QDs of multiple colors (n colors) and multiple intensity (m levels of intensity) are used, then the nanostructures may have a total number of unique identities or codes, which is equal to m to the exponent of n less one (mⁿ⁻¹). In addition, since the porous structure can be associated with additional payloads (e.g., fluorescent organic molecules), if there are Y number of additional fluorescent colors available, the total number of code can be Yx (mⁿ⁻¹).

In certain embodiments, the nanostructure (with or without bar-coding) is colored by being operably linked to a detectable agent. A detectable agent can be a fluorescent molecule, a chemo-luminescent molecule, a bio-luminescent molecule, a radioisotope, a MRI contrast agent, a CT contrast agent, an enzyme-substrate label, and/or a coloring agent etc.

Examples of fluorescent molecules include, without limitation, fluorescent compounds (fluorophores) which can include, but are not limited to: 1,5 IAEDANS; 1,8-ANS; 4-Methylumbelliferone; 5-carboxy-2,7-dichlorofiuorescein; 5-Carboxyfluorescein (5-FAM); 5-Carboxynaptho fluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AFPs—AutoFluorescent Protein—(Quantum Biotechnologies); Alexa® Fluor 350; Alexa® Fluor 405; Alexa® Fluor 500; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson™; Calcium Green; Calcium Green-1 Ca 2+ Dye; Calcium Green-2 Ca 2+; Calcium Green-5N Ca 2+; Calcium Green-C18 Ca 2+; Calcium Orange; Calcofluor White; Carboxy-X-rhodamine (5-ROX); Cascade Blue™; Cascade Yellow; Catecholamine; CCF2 (GeneBlazer); CFDA; Chlorophyll; Chromomycin A; Chromomycin A; CL-NERF; CMFDA; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′ DCFDA; DCFH (Dichlorodihydro fluorescein Diacetate); DDAO; DHR (Dihydrorhodamine 123); Di-4-ANEPPS; Di-8-ANEPPS (non-ratio); DiA (4-Di-16-ASP); Dichlorodihydro fluorescein Diacetate (DCFH); DiD-Lipophilic Tracer; DiD (DiIC18(5)); DIDS; Dihydrorhodamine 123 (DHR); Dil (DiIC18(3)); Dinitrophenol; DiO (DiOC18(3)); DiR; DiR (DiIC18(7)); DM-NERF (high pH); DNP; Dopamine; DTAF; DY-630-NHS; DY-635-NHS; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; FluoroGold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PYMPO; Pyrene; Pyronine; Pyronine B; Pyrozal Brilliant Flavin 7GF; QSY 7; Quinacrine Mustard; Red 613 [PE-TexasRed]; Resorufm; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; RhodamineBG; Rhodamine Green; Rhodamine Phallicidine; Rhodamine Phalloidine; Rhodamine Red; Rhodamine WT; Rose Bengal; R-phycocyanine; R-phycoerythrin (PE); S65A; S65C; S65L; S65T; SBFI; Serotonin; Sevron Brilliant Red 2B; Sevron Brilliant Red 4G; Sevron Brilliant Red B; Sevron Orange; Sevron Yellow L; SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thio flavin 5; Thioflavin S; Thioflavin TCN; Thiolyte; Thiozole Orange; Tinopol CBS (Calcofiuor White); TMR; TO-PRO-1; TO-PRO-3; TO-PRO-5; TOTO-1; TOTO-3; Tricolor (PE-Cy5); TRITC TetramethylRodaminelsoThioCyanate; True Blue; TruRed; Ultralite; Uranine B; Uvitex SFC; WW 781; X-Rhodamine; XRITC; Xylene Orange; Y66F; Y66H; Y66W; YO-PRO-1; YO-PRO-3; YOYO-1; YOYO-3, Sybr Green, Thiazole orange (interchelating dyes), fluorescent semiconductor nanostructures, lanthanides or combinations thereof

Examples of radioisotopes include, ¹²³I, ¹²⁴I, ¹²⁵I, ¹³¹I, ³⁵S, ³H, ¹¹¹I, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, ³²P, ¹⁸F, ²⁰¹Tl, ⁶⁷Ga, ¹³⁷Cs and other radioisotopes.

Examples of enzyme-substrate labels include, luciferases (e.g., firefly luciferase and bacterial luciferase), luciferin, 2,3-dihydrophthalazinedionesm, alate dehydrogenase, urease, peroxidase such as horseradish peroxidase (HRPO), alkaline phosphatase, galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Methods of Uses of the Nanocomposition

Another aspect of the present disclosure provides a method for modulating the behavior of a cell by contacting the cell with at least one cell-modulating agent operably linked to a nanostructure. The cell-modulating agent interacts with a molecule on the surface of the cell, and the interaction between the cell-modulating agent and the molecule modulates the behavior of the cell.

In some embodiments, the cell-modulating agent is a molecule (e.g., antibody or ligand) that specifically binds to a receptor on the surface of the cell so that the binding will lead to the change of function or property of the cell. The change of function or property of a cell can be determined using suitable methods known in the art, including, for example, observation using microscopy, cell counting, cell sorting, immuno-histochemistry, immuno-cell-chemistry, PCR, northern-blot, southern blot, western-blot (see, e.g., Julio E. Celis et al., Cell Biology, A Laboratory Handbook (3rd Ed.)). The cells whose behavior has been modulated can be isolated, enriched, or purified for further investigation or therapeutic application using the methods known in the art. In some preferred embodiments, the cell whose behavior has been modulated can be enriched by applying a magnetic field to pull down the cell. In such embodiments, the nanostructure operably linked to the cell-modulating agent comprises magnetic material. After the nanocomposition is administered to the cell and modulate its behavior, the cell specifically binds to the nanocomposition can be pull down by applying a magnetic field. In certain embodiments, the enriched cell can further be purified by redispersing the cell and applying a magnetic field to pull down the cell repeatedly. Alternatively, the nanocomposition comprises the cell-modulating agent does not comprise magnetic material. After the cell's behavior is modulated, a second nanocomposition that comprises an agent specifically recognizing the modulated cells is administered, and a magnetic field is applied to pull down the modulated cells. It is contemplated that the non-magnetic nanoparticle includes but is not limited to the nanoparticle that has been disclosed in U.S. Prov. Appl. 61/589,777 and U.S. patent application Ser. No. 12/460,007, as far as the non-magnetic nanoparticle is capable of carrying the cell-modulating agent.

In certain embodiments, the modulated cells being enriched, isolated or purified does not need to be processed to remove the nanocomposition before the cell is used for further investigation or therapeutic application.

In certain embodiments, methods of modulating cell behavior comprise the steps of contacting the cell with two or more cell-modulating agents, which act synergistically to modulate the behavior of a cell. The two or more cell-modulating agents can be operably linked to one nanostructure. Alternatively, the two or more cell-modulating agents can be operably linked to different nanostructure respectively.

In certain embodiments, methods for modulating cell are disclosed using a plurality of magnetic nanocomposition. The methods comprise the steps of contacting the cell with a first cell-modulating agent operably linked to a first nanostructure. The first nanostructure comprises a paramagnetic material. After the cell is modulated by the first cell-modulating agent, the cell is enriched by applying a strong magnetic field. The enriched cell is then further administered a second cell-modulating agent operably linked to a second nanostructure. The second nanostructure comprises a superparamagnetic material. After the cell is modulated by the second cell-modulating agent, the cell is then enriched by applying magnetic field within which only the cells binding to the second cell-modulating agent, but not the cells binding to the first cell-modulating agent are pulled down. Such method provides that the nanocomposition does not need to be removed before the modulated cells are used for further investigation or therapeutic application.

In certain embodiments, the method for modulating the behavior of a cell comprises the steps of administering nanocomposition to a subject, thus contacting the cells in vivo to the cell-modulating agent. For example, a nanocomposition comprising a vaccine can be administered to a subject to improve the subject's immunity to a particular disease.

In certain embodiments, the method for modulating the behavior of a cell comprises the steps of administering the modulated cells to a subject, and tracking the fate of the modulated cells within the subject. In such embodiments, the nanocomposition further comprises a detectable label operably linked to the nanostructure. For example, the detectable label can be a fluorescent molecule, a chemo-luminescent molecule, a bio-luminescent molecule, a radioisotope, a MRI contrast agent, a CT contrast agent, an enzyme-substrate label, or a coloring agent.

In certain embodiments, the method for modulating the behavior of a cell can be carried out when the molecule on the surface of the cell in a sample is at a sub-nanogram level.

In certain embodiments, the term “sub-nanogram level” refers to no more than 100 ng, 10 ng, ing or 0.1 ng of a molecule. For example, the sub-nanogram includes 0.01 ng, 0.02 ng. 0.03 ng, 0.04 ng, 0.05 ng, 0.06 ng, 0.07 ng, 0.08 ng, 0.09 ng, 0.1 ng, 0.2 ng, 0.3 ng, 0.4 ng, 0.5 ng, 0.6 ng, 0.7 ng, 0.8 ng, 0.9 ng, 1.0 ng, or any ranges between any of above mentioned level (e.g., between 0.01 ng and 100 ng, 0.01 ng and 10 ng, 0.01 ng and 1 ng, 0.01 ng and 0.1 ng).

In certain embodiments, the sub-nanogram level means no more than 1000 pM, 100 pM, 10 pM, 1 pM, 0.1 pM, 0.01 pM, 0.001 pM (=1 fM) or 0.0001 pM of an analyte. For example, the sub-nanogram includes 0.001 pM (=1 fM), 0.002 pM. 0.003 pM, 0.004 pM, 0.005 pM, 0.006 pM, 0.007 pM, 0.008 pM, 0.009 pM, 0.01 pM, 0.02 pM, 0.03 pM, 0.04 pM, 0.05 pM, 0.06 pM, 0.07 pM, 0.08 pM, 0.09 pM, 0.1 pM, 0.1 pM, 0.2 pM, 0.3 pM, 0.4 pM, 0.5 pM, 0.6 pM, 0.7 pM, 0.8 pM, 0.9 pM, 1 pM, 2 pM, 3 pM, 4 pM, 5 pM, 6 pM, 7 pM, 8 pM, 9 pM, 10 pM or any ranges between any of above mentioned level (e.g., between 0.0001 pM and 1000 pM, 0.0001 pM and 100 pM, 0.0001 pM and 10 pM, 0.0001 pM and 1 pM, 0.0001 pM and 0.1 pM, 0.0001 pM and 0.01 pM, 0.0001 pM and 0.001 pM).

In certain embodiments, the sub-nanogram level means a single cell, a plurality of cells (e.g., 2, 3, 4, 5, 6, 7, 8, 9, 10, 20, 30, 40, 50, 60, 70, 80, 90, 100, 200 cells) in a sample.

In certain embodiments, the method for modulating a cell further comprises enriching a population of said cell.

Another aspect of the present invention relates to a method for treating a disease in a subject. The method comprises contacting a cell with at least one cell-modulating agent operably linked to a nanostructure. The cell-modulating agent interacts with a molecule on the surface of the cell. The interaction between the cell-modulating agent and the molecule modulates a behavior of the cell. And modulated cells are then administered to the subject. In certain preferred embodiment, the disease being treated is cancer.

The term “subject”, as used in the description, relates to animals, preferably mammals, and more preferably, humans. The term “subject” does not aim to be limiting in any aspect, and can be of any age, sex and physical condition.

Methods for Preparing the Nanocomposition

Another aspect of the present disclosure relates to methods of forming a nanocomposition comprising a nanostructure and at least one cell-modulating agent operably linked to the nanostructure, wherein the cell-modulating agent can interact with a molecule on the surface of a cell, wherein the interaction between the cell-modulating agent and the molecule modulates a behavior of the cell. In certain embodiments, the cell-modulating agent and/or detectable label may be mixed with a readily formed nanostructure, e.g., in solution, dispersion, suspension, emulsion etc, to allow incorporation of the cell-modulating agent to the porous compartment of the nanostructure, or to allow conjugation of the cell-modulating agent to the functional groups on the nanostructure.

In certain embodiments, the cell-modulating agent may be introduced during or after the formation of the nanostructures. For example, when the nanostructure is formed through silanization process, the cell-modulating agent can be introduced to the silanization system, so as to allow the incorporation of the cell-modulating agent into the nanostructure during the silanization process. For another example, for a nanostructure having a surface reactive group (such as streptavidin), the cell-modulating agent comprises a binding partner to the reactive group (such as biotin) can be mixed with the nanostructure under conditions which facilitate the binding.

Methods for Preparing the Nanostructure

Another aspect of the present disclosure relates to methods of forming a nanostructure comprising at least one core nanoparticle with a coating. For example, the nanostructure is formed by coating or surrounding one or more core nanoparticle with a coating material such that the particle(s) is or are embedded in the coating material. For another example, the coating material is formed by crosslinking a precursor in the presence of a core nanoparticle, so that the nanoparticle is embedded in the crosslinked coating material.

In certain embodiments, the method further comprises introducing one or more functional groups within or on the surface of the nanostructure. The functional groups may be introduced during the formation of the coating material. For example, during the cross-linking process, precursors containing such functional groups can be added, in particular, during the ending stage of the cross-linking process. The functional groups may also be introduced after the formation of the nanostructure, for example, by introducing functional groups to the surface of the nanostructure by chemical modification. In certain embodiments, the functional groups are inherent in the nanostructure or in the coating material. The functional groups serve as linkage between the nanostructure and the cell-modulating agent. Examples of the functional groups include, but are not limited to amino, mercapto, carboxyl, phosphonate, biotin, streptavidin, avidin, hydroxyl, alkyl or other hydrophobic molecules, polyethylene glycol or other hydrophilic molecules, and photo cleavable, thermo cleavable or pH responsive linkers.

In certain embodiments, the method further comprises purifying the obtained nanostructure product. The purification may include use of dialysis, tangential flow filtration, diafiltration, or combinations thereof.

Methods for Preparing the Nanostructure Having a Low-Density Porous 3-D Structure

Another aspect of the present disclosure relates to methods of forming a nanostructure comprising at least one core nanoparticle with low-density, porous 3-D structure. For example, the nanostructure is formed by coating or surrounding one or more core nanoparticle with low density, porous 3-D structure such that the particle(s) is or are embedded in the 3-D structure.

The low-density, porous 3-D structure is formed by the depositing, or covering of the surface of the core nanoparticle through the assembly or cross-linking of silane-containing or silane-like molecules. The low density porous 3-D structure can be prepared by a silanization process on the surface of the core nanoparticles.

Silanization process includes, for example, the steps of crosslinking silicon-containing or silane-like molecules (e.g., alkoxysilanes such as amino-propyl-trimethoxysilane, mercapto-propyl-trimethoxysilane, or sodium silicate) under acidic or basic conditions.

In certain embodiments, an acidic or a basic catalyst is used in the crosslinking. Exemplary acid catalyst includes, without limitation, a protonic acid catalyst (e.g., nitric acid, acetic acid and sulphonic acids) and Lewis acid catalyst (e.g., boron trifluoride, boron trifluoride monoethylamine complex, boron trifluoride methanol complex, FeCl₃, AlCl₃, ZnCl₂, and ZnBr₂). Exemplary basic catalysts include, an amine or a quaternary ammonium compound such as tetramethyl ammonium hydroxide and ammonia hydroxide. The silanization process may include one or more stages, for example, a priming stage in which the 3-D structure starts to form, a growth stage in which a layer of siliceous structure is readily formed on the core nanoparticle and more are to be formed, and/or an ending stage in which the 3-D structure is about to be completed (e.g., the outer surface of the 3-D structure is about to be formed). During the silanization process, one or more silane-containing molecules can be added at different stages of the process. For example, in the priming stage, organosilanes such as aminopropyl trimethoxyl silane or mercaptopropyl trimethoxyl silane can be added to initiate the silanization on the core nanoparticle surface. For another example, silane molecules having fewer alkoxy groups (e.g., only 2 alkoxy groups) can be added to the reaction at the growth stage of silanization. For another example, at the ending stage of silanization, organo silane molecules with one or a variety of different functional groups may be added. These functional groups can be amino, carboxyl, mercapto, or phosphonate group, which can be further conjugated with other molecules, e.g., hydrophilic agent, a biologically active agent, a detectable label, an optical responsive group, electronic responsive group, magnetic responsive group, enzymatic responsive group or pH responsive group, or a binding partner, so as to allow further modification of the 3-D structure in terms of stability, solubility, biological compatibility, capability of being further conjugation or derivation, or affinity to payload. Alternatively, the functional groups can also be a group readily conjugated with other molecules (e.g., a group conjugated with biologically active agent, a thermal responsive molecule, an optical responsive molecule, an electronic responsive molecule, a magnetic responsive molecule, a pH responsive molecule, an enzymatic responsive molecule, a detectable label, or a binding partner such as biotin or avidin).

To control the formation of low density siliceous structure, the preparation further includes density reducing procedures such as introducing air bubbles in the reaction or formation, increasing reaction temperature, microwaving, sonicating, vertexing, labquakering, and/or adjusting the chemical composition of the reaction to adjust the degree of the crosslinking of the silane molecules. Without being bound to theory, it is believed that these procedures can help make the reaction medium homogeneous, well dispersed and promote the formation of low density porous 3-D structure with increased voids or porosity. In certain embodiments, the density reducing procedure comprises sonicating the reaction or formation mixture. The conditions of the sonicating procedure (e.g., duration) in the silanization process can be properly selected to produce a desired porosity in the resulting low density porous 3-D structure. For example, the sonicating can be applied throughout a certain stage of the silanization process. The duration of sonicating in a silanization stage may last for, e.g., at least 1 hour, 1.5 hours, 2 hours, 2.5 hours, 3 hours, 3.5 hours, 4 hours. In certain embodiments, sonicating is applied in each stage of the silanization process.

In certain embodiments, the density reducing procedures comprise introducing at least one alcohol to the reaction. In certain embodiments, the alcohol has at least 3 (e.g., at least 4, at least 5 or at least 6) carbon atoms. For example, the alcohol may have 3, 4, 5, 6, 7, 8, 9, 10, 11, 12 or more carbon atoms. In certain embodiments, the alcohol can be monohydric alcohols, or polyhydric alcohols. Illustrative examples of monohydric alcohols include, propanol, butanol, pentanol, hexyl alcohol, etc.

Illustrative examples of polyhydric alcohols include, propylene glycol, glycerol, threitol, xylitol, etc. In certain embodiments, the alcohol can have a saturated carbon chain or an unsaturated carbon chain. An alcohol having a saturated carbon chain can be represented as CₙH_{(2N+2)}O in chemical formula. In certain embodiments, n is no less than 3, or no less than 4, or no less than 5 (e.g., n=3, 4, 5, 6, 7, 8, 9, 10, 11, 12 or more). Alcohol with an unsaturated carbon chain has a double or a triple bond between two carbon atoms. In certain embodiments, the alcohol can be a cyclic alcohol, for example, cyclohexanol, inositol, or menthol.

In certain embodiments, the alcohol can have a straight carbon chain (e.g., n-propyl alcohol, n-butyl alcohol, n-pentyl alcohol, n-hexyl alcohol, etc) or a branched carbon chain (e.g., isopropyl alcohol, isobutyl alcohol, tert-butyl alcohol, etc). In certain embodiments, the alcohol is present in a volume fraction of about 30% to about 70% (e.g., about 30% to about 70%, about 30% to about 60%, about 30% to about 55%, about 40% to about 70%, about 45% to about 70%, about 40% to about 60%). In certain embodiments, the alcohol is present in volume fraction of around 50%) (e.g., around 45%, around 46%, around 47%, around 48%, around 49%, around 50%), around 51%, around 52%, around 53%, around 54%, around 55%, around 56%, around 57%, around 58%, around 59%, or around 60%,). In certain embodiments, the density reducing procedure comprises introducing air bubbles to the reaction. In certain embodiments, the air bubbles can be in constant presence during the reaction process. The air bubbles can be introduced to the reaction through any suitable methods, for example, by blowing bubbles to the reaction, or by introducing a gas-producing agent to the reaction mixture.

Other experimental conditions can also be optimized to provide for formation of a desired low density porous 3-D structure. Such experimental conditions include, for example, the concentration of the core nanoparticles, the concentration of the catalyst, the ratio of the concentration of the catalyst to the core nanoparticle, the temperature at which the low density siliceous structure is formed, or the molecular structure of the organosilanes.

The thickness of the low density porous 3-D structure, which directly correlates to the size of the nanostructure, could be controlled (e.g., from 1 nm to 1000 nm) by, for example, modifying the quantity of the silane-containing molecules (e.g., trialkoxysilane or sodium silicate), the reaction time, and time lapse between reaction steps and such kind of reaction parameters.

The thickness of the 3-D structure can be about 1 to 5 nm thick. In certain embodiments, the thickness can be about 1 to 10 nm thick. In certain embodiments, the thickness can be about 1 to 20 nm thick. In certain embodiments, the thickness can be about 1 to 30 nm thick. In certain embodiments, the thickness can be about 1 to 40 nm thick. In certain embodiments, the thickness can be about 1 to 50 nm thick. In certain embodiments, the thickness can be about 1 to 60 nm thick. In certain embodiments, the thickness can be about 1 to 100 nm thick. In certain embodiments, the thickness can be about 1 to 500 nm thick. In certain embodiments, the thickness can be about 1 to 1000 nm thick.

After the low-density, porous 3-D structure is formed on the surface of the core nanoparticle, the core nanoparticle is embedded in the 3-D structure. The resulting nanostructure can have a thickness (e.g., the longest dimension of the nanostructure or a diameter if the structure is a sphere) of about 1 to 1000 nm, 1 to 100 nm, or 1 to 10 nm. In another embodiment, the nanostructure can have a diameter of about 1 to 30 nm. In another embodiment, the nanostructure can have a diameter of about 500 nm. In another embodiment, the nanostructure can have a diameter of about 100 nm. In another embodiment, the nanostructure can have a diameter of about 50 nm. In another embodiment, the nanostructure can have a diameter of about 30 nm. In another embodiment, the nanostructure can have a diameter of about 10 nm.

The nanostructure having a low density 3-D structure prepared herein may be operably linked with one or more cell-modulating agent, using methods described herein and/or conventional methods known in the art. Optionally, the cell-modulating agent may be characterized as well, such as the amount of the cell-modulating agent.

### Example 1

### Preparation of Nanocompositions

Nanocompositions were prepared with superparamagnetic iron oxide nanoparticles with silanization encapsulation. Final concentration of nanocomposition was adjusted to be 1 mg/ml. 0.3 mg/ml of streptavidin molecules were covalently conjugated to nanocomposition through a crosslinker Sulfo-SMCC, after overnight incubation, nanocomposition-streptavidin conjugates were purified from the rest of the solution by magnetic separation.

Anti-CD3 (Clone OKT3), anti-CD28 (Clone28.2), or other costimulating antibodies were biotinlyated first following suggested protocol using commercial biotinylation kit (Thermo Scientific). The purified biotinylated-antibodies were mixed with streptavidin-nanocomposition at the defined antibody/nanocomposition quantity and react overnight, then magnetically purified to form the needed antibody-conjugated nanocompositions.

### Example 2

### Expansion of T Cells Using Anti-CD3/Anti-CD28 Conjugated Nanocompositions

It has been reported that immobilized anti-CD3 and anti-CD28 antibodies can simultaneously deliver a signal and a co-stimulatory signal to stimulate proliferation of T cells (Baroja et al (1989), Cellular Immunology, 120: 205-217). In WO09429436A1 solid phase surfaces such as culture dishes and beads are used to immobilize the anti-CD3 and anti-CD28 antibodies. Regularly, the immobilization on beads is performed on DynaBeads®M-450 having a size of 4.5 um in diameter.

US2008/0317724A1 discloses that the spatial presentation of signal molecules can dramatically affect the response of T cells to those signal molecules. For example, when anti-CD3 and anti-CD28 antibodies are placed on separate predefined regions of a substrate, T cells incubated on the substrate secrete different amounts of interleukin-2 and/or exhibit spikes in calcium, depending not only on the types but also on the spacing of these signal molecules. For example, a pattern was generated with anti-CD3 and anti-CD28 antibodies, where anti-CD3 antibodies occupied a central feature surrounded by satellite features of anti-CD28 antibodies that were spaced about 1 to 2 microns from the central anti-CD3 feature. When the anti-CD28 antibody features were spaced about 1 to 2 microns apart, the T cell secretion of interleukin-2 (IL-2) was enhanced compared to when the anti-CD3 and anti-CD28 antibodies were presented together to the T cells in “co-localized” features.

Erin R Steenblock and Tarek M Fahmy (Molecular Therapy vol. 16 no. 4, 765-772 April 2008) reported using solid-surface nanoparticles (130 nm) and show that these nanoparticle stimulate T cells weaker than microparticles (8 um). The authors stated that these findings are supported by those of previous reports (Mescher, MF (1992). J Immunol 149: 2402-2405.), demonstrating that micron-sized particles, which are close in size to T cells, provide optimal T-cell stimulation. Mesher's study demonstrated the critical importance of a large, continuous surface contact area for effective CTL activation. Using class I alloantigen immobilized on latex microspheres, particle sizes of 4 to 5 microns were found to provide an optimum stimulus. Below 4 microns, responses decreased rapidly with decreasing particle size, and large numbers of small particles could not compensate for suboptimal size. U.S. Pat. No. 8,012,750B2 discloses a biodegradable device for activating T-cells. According to U.S. Pat. No. 8,012,750B2 nanospheres do not provide enough cross-linking to activate naive T-cells and thus can only be used with previously activated T-cells. Again, experimental data were generated with spheres co-immobilized with anti-CD3 and anti-CD28 antibodies ranging in size from 4 to 24 microns with a mean of 7 microns.

In the present Example, CD4+ T cells were purified from fresh or frozen human PBMC by magnetic separation using anti-CD4 antibody conjugated nanostructure. Similarly, CD8+ T cells were prepared from fresh or frozen human PBMC by magnetic purification using anti-CD8 antibody conjugated nanostructure. CD4+ or CD8+ T cells were plated with 2-4×10⁶ cells/ml. This counted as Day 0. On Day 1, anti-CD3/anti-CD28 conjugated nanocomposition were added to the cells. On Day 3, IL-2 and more medium was added to the cells. On Day 5, cells were counted, medium were changed with IL-2 added. On Day 7 and 10, IL-2 was added. On day 12, cell numbers were counted.

As shown in Table 1-4, nanocomposition with various concentration of anti-CD3/anti-CD28 antibodies conjugated stimulate the expansion of CD4+ or CD8+ T cells.

TABLE 1
Expansion of CD4+ T cells

| Concentration | Cell Number (10⁶) | | |
|---|---|---|---|
| Anti-CD3/anti-CD28 | Day 0 | Day 5 | Day 12 |
| 1 ug/10 ug | 1 | 1.1 | 5.8 |
| 4 ug/40 ug | 1 | 1.3 | 13.0 |
| 5 ug/5 ug | 1 | 1 | 12.3 |
| 3.75 ug/3.75 ug | 1 | 1.3 | 7.0 |
| Control# | 1 | 0.9 | 0.2 |

#Control: nanostructure without antibody conjugation

TABLE 2
Expansion of CD8+ T cells

| Concentration | Cell Number (10⁶) | | |
|---|---|---|---|
| Anti-CD3/anti-CD28 | Day 0 | Day 5 | Day 12 |
| 1 ug/10 ug | 1 | 0.8 | 3.5 |
| 4 ug/40 ug | 1 | 1.2 | 2.3 |
| 5 ug/5 ug | 1 | 1.9 | 4.0 |
| 3.75 ug/3.75 ug | 1 | 1.1 | 1.4 |

TABLE 3
Re-stimulation of CD4+ T cells

| Concentration | Cell Number (10⁶) | | |
|---|---|---|---|
| Anti-CD3/anti-CD28 | Day 12 | Day 14 | Day 17 |
| 5 ug/5 ug | 1 | 2.2 | 6.3 |
| 3.75 ug/3.75 ug | 1 | 1.7 | 4.2 |

TABLE 4
Re-stimulation of CD8+ T cells

| Concentration | Cell Number (10⁶) | | |
|---|---|---|---|
| Anti-CD3/anti-CD28 | Day 12 | Day 14 | Day 17 |
| 5 ug/5 ug | 1 | 1.5 | 1.9 |
| 3.75 ug/3.75 ug | 1 | 1.7 | 3.8 |

### Example 3

### Comparison of Nanocomposition to Dynabeads®

Streptavidin conjugated magnetic low density nanostructures at 1 mg/ml concentration were mixed with 1 ug/ml biotinylated anti-CD3 antibody and 10 ug/ml biotinylated anti-CD28 antibody to prepare anti-CD3/anti-CD28 conjugated nanocomposition. Anti-CD3 antibody were added to the nanostructure first and incubated for 30 min, subsequently anti-CD28 antibodies were added and the solution was left on a rotator at 4° C. overnight. On the next day, nanostructure-anti CD3/CD28 antibody conjugates were purified from the rest of solution using magnetic separation, and redispersed in PBS buffer, ready to use. Fresh human PBMC without purification were adjusted to 10⁶ cells/ml. 50 ul of 1 mg/ml of anti-CD3/anti-CD28 conjugated nanocomposition were added to the cells. Dynabeads® (Life Technologies) were used following its protocol at 1:1 beads/T cells ratio. Used 25 ul washed beads/ml of 10⁶ T cells.

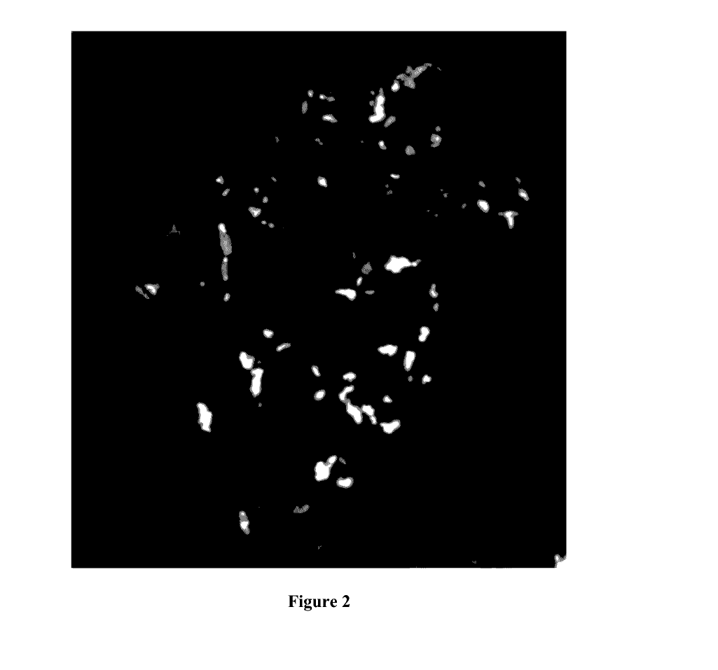

As shown in FIG. 2 and Table 5, anti-CD3/anti-CD28 conjugated nanocomposition shows higher T-cell stimulation (expression of CD69) as compared to anti-CD3/anti-CD28 conjugated Dynabeads®. Various T cell subsets can have different activation requirements. In particular, naïve T cells are difficult to activate in the absence of accessory cells. Our results show that all T cell subsets can be activated well by nanocompositions. As shown in FIG. 2 and Table 5, more CD4+ T cells, CD4+ naïve T cells, CD4+ central memory T cells, CD+ effector memory T cells were activated in the presence of anti-CD3/anti-CD28 conjugated nanocomposition than in the presence of anti-CD3/anti-CD28 conjugated Dynabeads®.

TABLE 5
Stimulation of CD4+ T cells using anti-CD3/anti-CD28 antibody
conjugated nanocomposition.

| | Percentage of cells activated | | |
|---|---|---|---|
| | | Conjugated | Conjugated |
| T Cell type | Control# | nanocomposition | Dynabeads ® |
| CD4+ total | 3.29 | 76.32 | 56.86 |
| CD4+ naïve | 2.48 | 87.36 | 69.53 |
| CD4+ central memory | 6.24 | 61.70 | 46.90 |
| CD4+ effector memory | 5.16 | 67.93 | 54.03 |

#Control: nanostructure without antibody conjugation

### Example 4

### Isolation and Identification of Circulating Tumor Cells Using Nanocomposition

Nanostructures are prepared with both magnetic and fluorescent property by encapsulating SPIO and quantum dots in a silanization processing. 1 mg/ml multifunctional fluorescent magnetic nanostructures were conjugated with 0.3 mg/ml streptavidin through a crosslinker sulfo-SMCC. After magnetic separation, purified nanostructure-streptavidin conjugates are dispersed in PBS buffer. Anti EpCAM antibody or anti CD19 antibody were biotinlylated using commerical biotinylation kit following standard protocol. 1 mg/ml nanostructure-streptavidin were mixed with 20 ug/ml biotin-anti-EpCAM or 20 ug/ml biotin-anti-CD19, respectively, after overnight incubation at 4° C., nanostructure-anti EpCAM or nanostructure-anti CD19 was magnetically separated and purified. Final antibody conjugated nanocompostions were stocked in PBS buffer at 1 mg/ml concentration. For cell separation, 20 to 500 ul of antibody conjugated nanocompositions were mixed with 10 to 1000 spiking cancer cells with CFSE or CMTMR pre-stain in whole blood samples at a volume of 0.5 to 7.5 ml. After incubating for 1 hour, nanocomposition captured cells were separated from the rest of whole blood sample using a magnet.

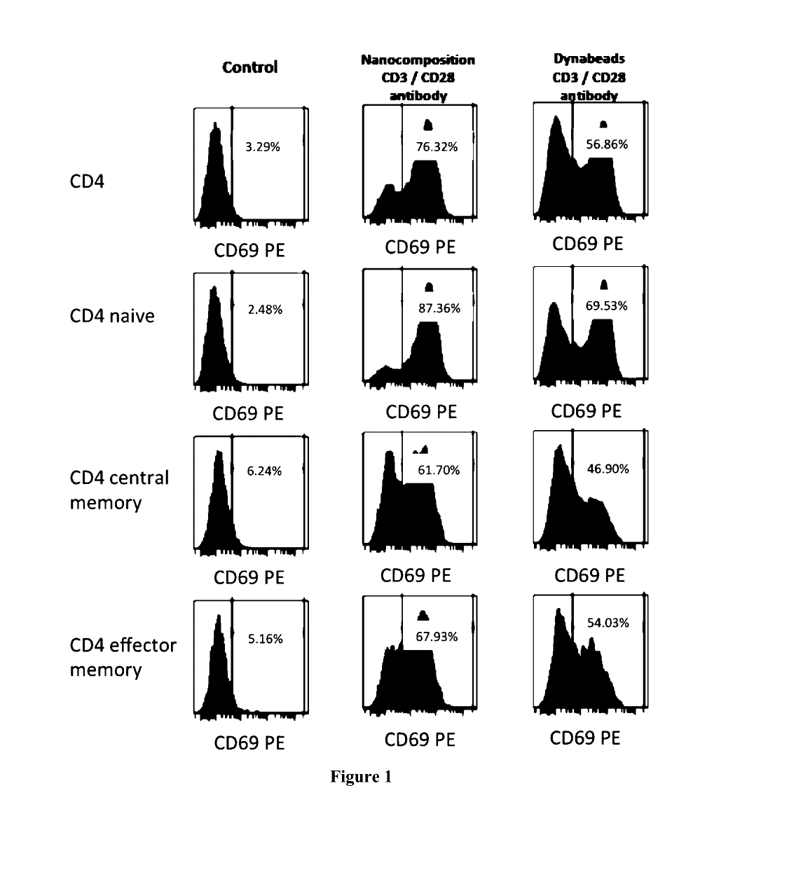

As shown in FIG. 1, captured cells were of high purity and high yield (both >90%). The fluorescent color identified the cell type and indicated cell surface molecular location and function. Two different types of circulating tumor cells interacted and isolated with nancompositions of multifunctional fluorescent and magnetic property from a whole blood sample. The specific interaction is from nanostructure surface conjugated antibody and cell surface molecules. The red fluorescence nanostructure (615 nm emission) has anti-EpCAM antibody on surface, they interacted with H1650 cells (CF SE stained green). The green fluorescence nanostructure (535 nm emission) has anti-CD 19 on surface, they interacted with Oc1-Ly8 (CMTMR stained cherry). These multifunctional nanocompositions not only interacted with cells, but also identified the cell type or cell surface marker via fluorescence signal.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A nanocomposition for cell modulation comprising:
a nanostructure comprising at least one nanoparticle;
at least one cell-modulating agent operably linked to the nanostructure;
wherein the cell-modulating agent is capable of interacting with a molecule on the surface of a cell.

2. The nanocomposition of claim 1, wherein the nanoparticle comprises a superparamagnetic iron oxide (SPIO) nanoparticle, or a non-SPIO nanoparticle.

3. The nanocomposition of claim 1, wherein the nanoparticle has a diameter ranging from about 1 nm to about 900 nm.

4. The nanocomposition of claim 1, wherein the nanostructure further comprises a low density, porous 3-D structure, wherein said at least one nanoparticle is embedded in the 3-D structure.

5. The nanocomposition of claim 4, wherein the low density, porous 3-D structure has a thickness ranging from 1 nm to 500 nm.

6. The nanocomposition of claim 1, wherein the cell-modulating agent is selected from the group consisting of an antibody, a ligand, a peptide, a cytokine, a hormone, a nucleic acid, a vitamin, a metabolite collagen, a polysaccharide, a glycosaminoglycan, an extracellular matrix composition and a combination thereof.

7. The nanocomposition of claim 1, wherein the cell-modulating agent is selected from the group consisting of an anti-CD3 antibody, an anti-CD28 antibody, an anti-CD81 antibody, a CD28 ligand, an anti-CD5 antibody, an anti-CD4 antibody, an anti-CD8 antibody, an anti-CTLA-4 antibody, an anti-PD-1 antibody, and anti-PD-L1 antibody, an anti-CD278 antibody, an anti-CD27L antibody, an anti-CD137 antibody, a CD137 ligand protein, an anti-CD30L antibody, an IL-2, an IL-2 receptor antibody, a IL-15 protein, a IL-15 receptor antibody, an IL-12, an IL-12 receptor antibody, an IL-1, an IL-1 receptor antibody, an IFN-gamma, an IFN-gamma receptor antibody, an TNF-alpha, an TNF-alpha receptor antibody, an IL-4, an IL-4 receptor antibody, an IL-10, an IL-10 receptor antibody and any combination thereof.

8. The nanocomposition of claim 1, wherein the molecule on the surface of the cell is a receptor of the cell.

9. The nanocomposition of claim 1, wherein the cell is a T cell, a natural killer cell or a stem cell.

10. The nanocomposition of claim 9, wherein the cell is a CD4+ or CD8+ T cell.

11. The nanocomposition of claim 9, wherein the stem cell is an embryonic stem cell.

12. The nanocomposition of claim 9, wherein the cell comprises a chimeric antigen receptor.

13. The nanocomposition of claim 1, wherein the cell-modulating agent interacts with the cell so as to modulate a behavior of the cell.

14. The nanocomposition of claim 13, wherein the behavior of the cell is proliferation or differentiation.

15. The nanocomposition of claim 1, further comprising a detectable label operably linked to the nanostructure, said detectable label is selected from the group consisting of a fluorescent molecule, a chemo-luminescent molecule, a bio-luminescent molecule, a radioisotope, a MRI contrast agent, a CT contrast agent, an enzyme-substrate label, and a coloring agent.

16. A method for modulating a cell comprising contacting the cell with a nanocomposition, said nanocomposition comprising:
a population of nanostructures, each nanostructure comprising at least one nanoparticle;
one or more cell-modulating agents operably linked to the nanostructures;
wherein the cell-modulating agent interacts with a molecule on the surface of the cell, and wherein the interaction between the cell-modulating agent and the molecule modulates a behavior of the cell.

17. The method of claim 16, wherein at least one first and one second cell-modulating agents are operably linked to the same nanostructure.

18. The method of claim 16, wherein at least one first and one second cell-modulating agents are operably linked to separate nanostructures.

19. The method of claim 16, wherein the cell is a T cell, and wherein said cell-modulating agents provide activation signals to the T cell.

20. The method of claim 19, wherein the cell-modulating agent is selected from the group consisting of an anti-CD3 antibody, an anti-CD28 antibody, an anti-CD81 antibody, a CD28 ligand, an anti-CD5 antibody, an anti-CD4 antibody, an anti-CD8 antibody, an anti-CTLA-4 antibody, an anti-PD-1 antibody, and anti-PD-L1 antibody, an anti-CD278 antibody, an anti-CD27L antibody, an anti-CD137 antibody, a CD137 ligand protein, an anti-CD30L antibody, an IL-2, an IL-2 receptor antibody, a IL-15 protein, a IL-15 receptor antibody, an IL-12, an IL-12 receptor antibody, an IL-1, an IL-1 receptor antibody, an IFN-gamma, an IFN-gamma receptor antibody, an TNF-alpha, an TNF-alpha receptor antibody, an IL-4, an IL-4 receptor antibody, an IL-10, an IL-10 receptor antibody and any combination thereof.
